(19) United States
(12) Patent Application Publication
(10) Pub. No.: US 2015/0246201 A1
(43) Pub. Date: Sep. 3, 2015

(54) BRIDLE SYSTEM FOR PLACING AND SECURING A NASAL TUBE IN A PATIENT

(71) Applicants: David S. KIRN, Lexington, KY (US); Edward F. KIRN, Lexington, KY (US)

(72) Inventors: David S. Kirn, Lexington, KY (US); Edward F. Kirn, Lexington, KY (US)

(21) Appl. No.: 14/438,675

(22) PCT Filed: Oct. 24, 2013
(86) PCT No.: PCT/US13/66498

§ 371 Date: Apr. 27, 2015

Related U.S. Application Data
(60) Provisional application No. 61/718,424, filed on Oct. 25, 2012.

(51) Int. Cl. A61M 16/06

(52) U.S. Cl. CPC A61M 16/0688; A61M 16/0666

(57) ABSTRACT

A bridle system for placing and securing a medical tube in a patient includes a flexible member having a magnet attached to a first end for insertion into a patient's nose, and a magnetic probe for insertion into a second nare of the nose. The magnetic probe attracts the flexible member magnet to assist in retrieving the first end of the flexible member through the second nare of the nose. The system further includes a receiver for securing the medical tube and the flexible member externally, adjacent the nose to prevent dislodgement by the patient or otherwise. The receiver includes a main body and a cantilever arm extending from the main body, forming an arcuate recess for receiving the tube.

This application claims the benefit of U.S. Provisional Patent Application No. 61/718,424, filed Oct. 25, 2012, the disclosure of which is incorporated herein by reference.

### FIELD OF THE INVENTION

The present invention relates generally to systems for placing and securing a medical tube in a patient; and more particularly to such a system which utilizes magnets in the placement of a bridle used in combination with a receiver to secure the tube.

### BACKGROUND OF THE INVENTION

Many different systems and related methods have been used to place nasal tubes, such as feeding tubes, nasogastric tubes, and nasotracheal tubes into patients, and to secure the tubes once placed. One such system described in U.S. Pat. Nos. 6,631,715 and 6,837,237 is a magnetic nasal tube bridle system. This system utilizes a flexible member that is looped around a patient's nasal septum and secured with the medical tube inside a receiver in order to prevent removal of the tube by the patient. More specifically, magnets are used during insertion of the flexible member in the patient such that the flexible member is looped around the nasal septum of the patient. The ends of the looped flexible material are secured with the medical tube using a receiver. The receiver is a two-part, hinged device which clamps one or both ends of the flexible material and the medical tube in a central channel, or separate channels, formed in the receiver.

Given the design of the receiver, the receiver, or more specifically the inner channels of the receiver, must be sized in accordance with an outer diameter of the medical tube being retained because the receiver channel must secure the tube without deforming the tube causing blockages, or allowing the tube to slide. This is particularly difficult given the fact that the receiver is often utilized in environments which are moist due to the presence of oral and nasal secretions. If not properly sized, the patient could simply slide the tube through the receiver despite the nasal bridle which is designed to anchor the tube and prevent unintended removal thereof.

Given these requirements and obstacles, multiple receiver sizes are manufactured to accommodate the multiple sizes of medical tubes and the user must select an appropriate size receiver to ensure proper retention of the tube. Accordingly, a need exists for a universal receiver that can accommodate multiple tube sizes, including non-standard tube sizes such as are commonly utilized in pediatric, infant, and neonatal patients.

Even more, such a universal receiver needs to have overall smaller dimensions than existing receiver models to support use with the pediatric, infant, and neonatal applications while still accommodating multiple size tubes and ensuring proper retention of the tube by the receiver. The present invention meets these requirements by providing a receiver for use with a bridle system that is able to secure multiple tube diameters, even in moist conditions, while maintaining a smaller overall size. This provides additional comfort for all patients and allows the invention to be utilized with the non-standard tube sizes such as are commonly utilized in pediatric, infant, and neonatal patients.

Additional methods for securing nasal tubes include attaching the tubes directly to the skin of the recipient utilizing an adhesive such as tape or the like, as described in U.S. Pat. Nos. 4,114,626 and 4,282,871, or sutures. All of these methods which rely on an adhesive positioned on the patient to secure the feeding tubes have in common the disadvantages of being uncomfortable for the patient, gradually losing adhesion over time, risking injury to the underlying skin, or causing pressure necrosis of the nose itself from holding the tube tightly opposed to the nose.

Thus, as demonstrated by the limitations and disadvantages of the prior art methods for placing and securing nasal tubes in patients, there is a need identified for an improved bridle system and related method for placing and securing nasal tubes which allows rapid, easy looping of a bridle around the posterior nasal septum with minimal patient discomfort and no risk of mandibular dislocation. An additional need exists for a universal system and receiver capable of use with any type and/or size of nasal tube, including non-standard tube sizes such as are commonly utilized in pediatric, infant, and neonatal patients, in order to limit the amount of storage space required in the medical facility. All of these features are provided by the following invention.

### SUMMARY OF THE INVENTION

The present invention meets these needs by providing an improved receiver for securing a medical tube in a patient and a flexible member positioned around the nasal septum of the patient such that a first end of the flexible member extends from a first nare of the patient and a second end of the flexible member extends from a second nare of the patient. The receiver includes a main body securing at least one of the ends of the flexible member and having a first arcuate recess formed in an exterior surface of the main body for receiving the tube, and a cantilever arm extending from the main body and having a second arcuate recess formed in a first exterior surface of the cantilever arm for receiving the tube, the second arcuate recess substantially extending the first arcuate recess.

In another possible embodiment, the second arcuate recess has an opening, along an axial direction thereof, at least portions of the opening are smaller than an outer diameter of the tube for securing the tube in said recess.

In yet another possible embodiment, the receiver may include an adhesive tape having a first portion secured to the main body and a second portion having a backing thereon covering an adhesive such that the backing may be removed to expose the adhesive for adhering to at least the main body and the tube. Also, the first portion of the adhesive tape may be secured within the cantilever arm.

In still another possible embodiment, the first portion of the adhesive tape may be adhered to a second exterior surface of the cantilever arm.

In another possible embodiment, the first arcuate recess has an opening, along an axial direction thereof, at least portions of the opening being smaller than an outer diameter of the tube for securing the tube in the recess.

In one other possible embodiment, the first end of the flexible member extending from the first nare of the patient may be secured within the main body prior to insertion of the second end of the flexible member into the second nare of the patient. Even more, the flexible member may be molded within the main body.

In another possible embodiment, the second arcuate recess may have an opening, along an axial direction thereof, at least portions of the opening are smaller than an outer diameter of the tube for securing the tube in the recess.

The present invention further meets the stated needs by providing a system for placing and securing a nasal tube in a patient. The system includes a flexible member having first and second ends, the first end for insertion into a first nare of the patient, a magnet secured at the first end, a magnetic probe for insertion into a second nare of the patient to retrieve the magnet and the first end of the flexible member, and a receiver. The receiver has a main body securing at least one of the ends of the flexible member and having a first arcuate recess formed in an exterior surface of the main body for receiving the tube, and a cantilever arm extending from the main body and having a second arcuate recess formed in a first exterior surface of the cantilever arm for receiving the tube. The second arcuate recess substantially extends the first arcuate recess.

In another possible embodiment, the at least one of the first and second arcuate recesses has an opening, along an axial direction thereof, at least portions of the opening being smaller than an outer diameter of the tube for securing the tube in the recess.

In yet another possible embodiment, the first end of the flexible member extending from the first nare of the patient is secured within the main body prior to insertion of the second end of the flexible member into the second nare of the patient.

In still another possible embodiment, the flexible member is molded within the main body prior to insertion of the second end of the flexible member into the second nare of the patient.

In accordance with still another aspect of the present invention, a method for placing and securing a tube in a patient is provided. The method includes the steps of positioning the tube in the patient such that a portion of the tube extends from the patient, inserting an end portion of a flexible member into a first nare of the patient, the end portion having a magnet secured thereto, inserting a probe into a second nare of the nose of the patient for attracting the magnet, removing the probe from the second nare of the nose of the patient thereby retrieving the end portion of the flexible member through the second nare of the nose of the patient, positioning the portion of the tube extending from the patient within a recess formed in a receiver, the recess formed by a main body and a cantilever arm of a receiver, and securing the portion of the tube extending from the patient within the recess and against the receiver using an adhesive tape. The adhesive tape has a first portion secured to a first surface of the receiver and a second portion for adhering to the portion of the tube extending from the patient and a second surface of the receiver.

In accordance with another possible embodiment, the recess may have an opening along an axial direction thereof, wherein at least portions of the opening are smaller than an outer diameter of the tube for securing the tube in the recess. In yet another, the first end of the flexible member extending from the first nare of the patient may be molded within the main body prior to insertion of the second end of the flexible member into the second nare of the patient.

The Summary and Abstract sections may set forth one or more but not all exemplary embodiments of the present invention as contemplated by the inventors, and thus, are not intended to limit the present invention and the appended claims in any way.

Additional advantages and other novel features of the invention will be set forth in part in the description that follows and in part will become apparent to those skilled in the art upon examination of the following or may be learned with the practice of the invention. The objects and advantages of the invention may be realized and attained by means of the instrumentalities and combinations particularly pointed out in the appended claims.

### BRIEF DESCRIPTION OF THE DRAWINGS

The accompanying drawings incorporated in and forming a part of the specification, illustrate several aspects of the present invention, and together with the description serve to explain the principles of the invention. In the drawings:

FIG. 1 is a perspective view of a receiver for securing a nasal tube and ends of a flexible member;

FIG. 2 is a side elevational view of the receiver having an arcuate recess formed therein and the nasal tube in position to be secured by an adhesive tape secured within the receiver;

FIG. 3 is a perspective view of a flexible member for securing a nasal tube and having a magnet secured at a first end thereof;

FIG. 4 is a perspective view of a magnetic probe for retrieving the magnet and the flexible member;

FIG. 5 is a perspective view of a receiver for securing the nasal tube having an opening along an axial direction thereof that is smaller than an outer diameter of the nasal tube;

FIG. 6 is a side elevational view of an alternate embodiment of the receiver having an arcuate recess formed therein and the nasal tube in position to be secured by an adhesive tape adhered to the receiver;

FIG. 7 is a front elevational view of the receiver having an arcuate recess formed therein and the nasal tube secured in position by the adhesive tape;

FIG. 8 is a front elevational view of the receiver including the nasal tube in position and secured by the adhesive tape;

FIG. 9 is a partial cross-sectional view showing the patient's nasal septum, the nasal tube inserted in the patient, the flexible member positioned around the nasal septum, and the receiver securing the tube and flexible member ends; and

FIG. 10a-10d are partial cross-sectional views showing: (a) the initial insertion of the first end portion of the flexible member into a first nare; (b) the first end portion of the flexible member inserted an average distance to a point just beyond the choanal aperture and the initial insertion of a magnetic probe into a second nare; (c) the removal of the probe magnetically coupled to the flexible member from the second nare of the nose; and (d) the flexible member and the nasal tube after placement in the patient, and application of the receiver.

Reference will now be made in detail to the present preferred embodiment of the invention, an example of which is illustrated in the accompanying drawings.

### DETAILED DESCRIPTION OF THE PREFERRED EMBODIMENT

In the following detailed description of the illustrated embodiments, reference is made to the accompanying drawings that form a part hereof, and in which is shown by way of illustration, specific embodiments in which the invention may be practiced. These embodiments are described in sufficient detail to enable those skilled in the art to practice the invention and like numerals represent like details in the various figures. Also, it is to be understood that other embodiments may be utilized and that process, mechanical, electrical, arrangement, software and/or other changes may be made without departing from the scope of the present invention. In accordance with the invention, methods, systems, and devices are hereinafter described for use in placing and securing a tube in a patient.

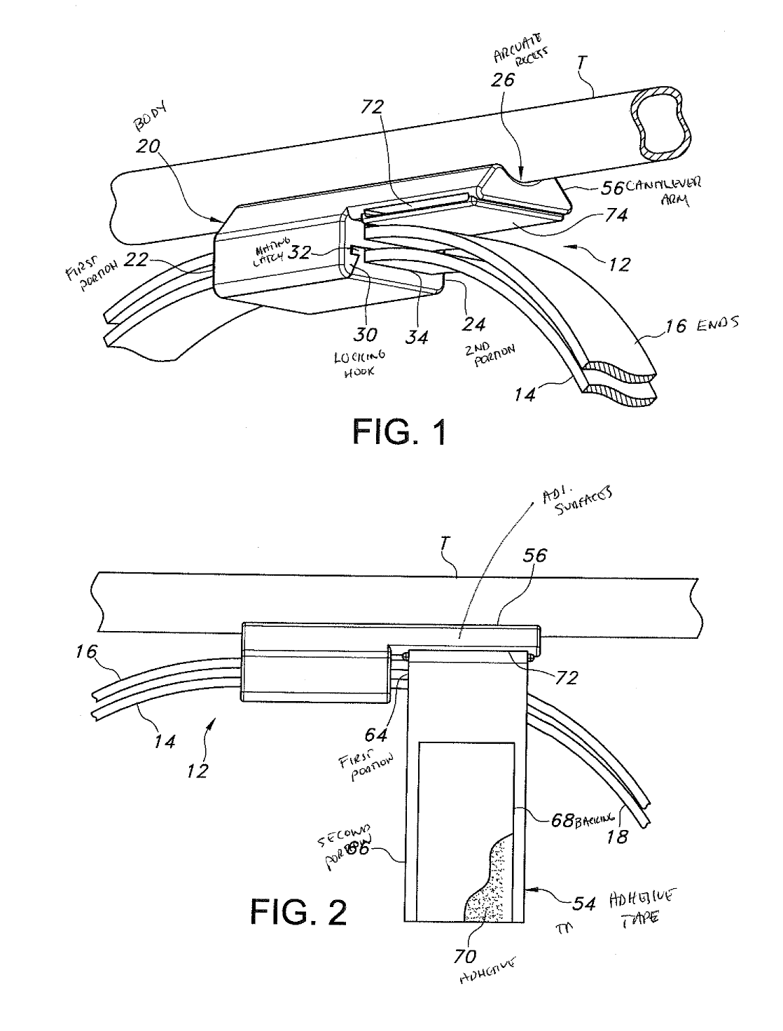

With reference to the perspective view of FIG. 1, there is shown an embodiment of a receiver 12 for securing a nasal tube T and end portions 14 and 16 of a flexible member 18 positioned within a patient. The receiver 12 consists of a molded plastic main body 20 having first and second members, or portions 22 and 24, for securing the end portions 14, 16 of the flexible member 18. In this embodiment, end portion 16 is permanently attached to main body 20 by molding, gluing, or otherwise securing. The other end portion 14 is secured by the main body 20 as described in detail below. The receiver 12 further includes an arcuate recess 26 formed in an exterior surface of main body first portion 22 for receiving the tube T. In accordance with an important aspect of the present invention, securing the tube along the exterior of the receiver 12 allows the receiver to be smaller than previous receivers that utilize an internal channel or the like for securing a tube therein. Even more, the smaller overall size receiver design is less intrusive to the patient and allows for use with pediatric, infant, and neonatal patients. Additional advantages of the present receiver 12 include rounded or contoured exterior surfaces which provide a smooth contact surface for the patient to prevent irritation.

As further shown in FIG. 1, the main body portions 22 and 24 are pivotally connected by a living hinge 28 (see FIG. 7) formed during molding of the receiver 12, but other means of connecting the main body portions may be utilized in accordance with the present invention. For example, the main body portions may simply snap one into the other without being pivotally connected. Integrally formed snap-type locking hook 30 extends from main body member 22 and a mating latch 32 is formed in member 24 for firmly securing the members together.

As shown in FIG. 2, the end portion 14 of flexible member 18 is secured between main body portions 22 and 24 following placement of the nasal tube T in the patient. More specifically, end portion 14 of flexible member 18 is secured within a recess 34 formed in at least one of the main body portions 22 and 24. The recess 34, as best shown in FIG. 7, may be completely formed in main body portion 22, half formed in main body portion 22 and half in main body portion 24, or more or less than half in each main body portion. Similarly, the shape and/or depth of recess 34 can be any shape or depth so long as one, or both, of end portions 14, 16 of flexible member 18 can be held securely therein. In accordance with the broad teaching of the present invention, the recess 34 is not even required for securing the end portions 14, 16 of flexible member 18 between main body portions 22 and 24. The flexible member 18 may be a generally flat, shoe-string shaped material, a tube-shaped material, or otherwise so long as the material is sufficiently compressible to be held between main body portions 22 and 24.

In fact, the flexible member 18 may be constructed of any material suitable for insertion into and retrieval from the patient. For example, the flexible member 18 may include differing materials, including a first tubular portion that itself is inserted and retrieved from the patient. In such an arrangement, a second flatter portion of the flexible member 18 would simply be pulled through the patient's nares around the septum by the first tubular portion, separated from the originally inserted first tubular portion, and the ends of the flexible member, comprising the second flatter portion, would then be secured by the receiver 12.

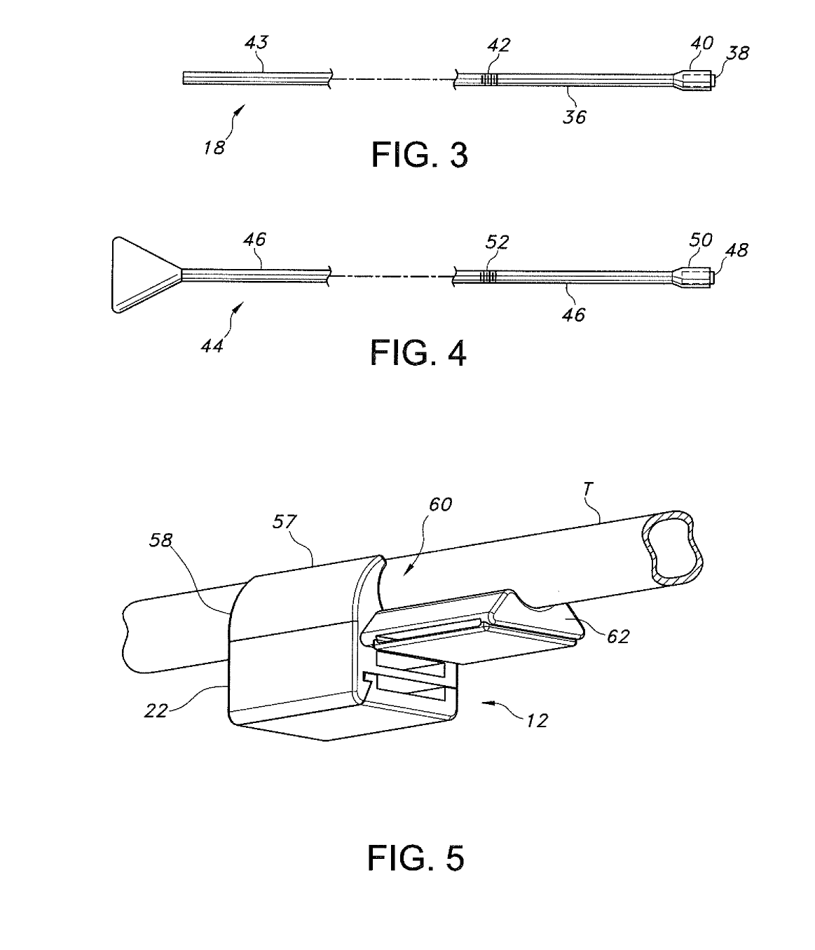

In the embodiment shown in FIG. 3, however, the flexible member 18 consists of a flexible tube portion 36 having a magnet 38, for example a powerful rare-earth magnet or a ferromagnetic magnet, attached to an end portion 40 of the flexible member, and a flatter portion 43. Preferably, the end portion 40 of the flexible member 18 is slid over the magnet 38 such that the magnet is substantially within the end portion 40. Alternatively, the flexible member 18 may be molded around the magnet 38 thereby securing the magnet in an integrated manner within the tube. Advantageously, either embodiment provides for smooth passage of the magnet 38 during its insertion and retrieval from the patient. Of course, other methods of securing the magnet to the end portion of the flexible tube 18, such as adhesives, tapes, etc., and any type of magnetic material and/or shape of magnet may be utilized in accordance with the present invention. In this embodiment, the flatter portion 43 of the flexible member 18 may be a polyester tape that is glued or otherwise affixed to the flexible tube portion 36. After insertion into the patient, all or some of the flexible tube portion 36 may be removed leaving the flatter portion 43 for use in securing the tube T.

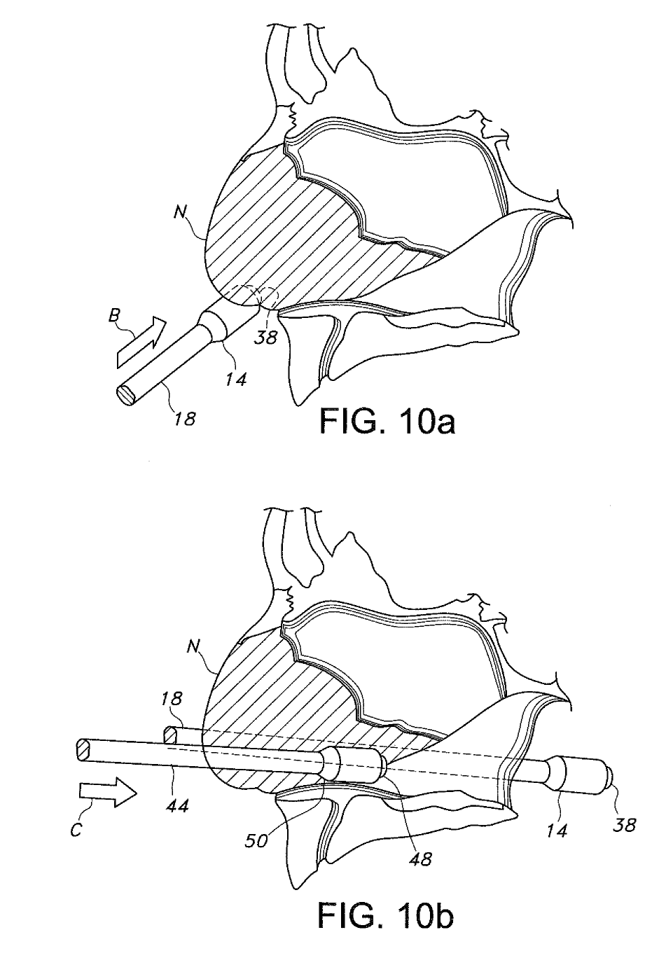

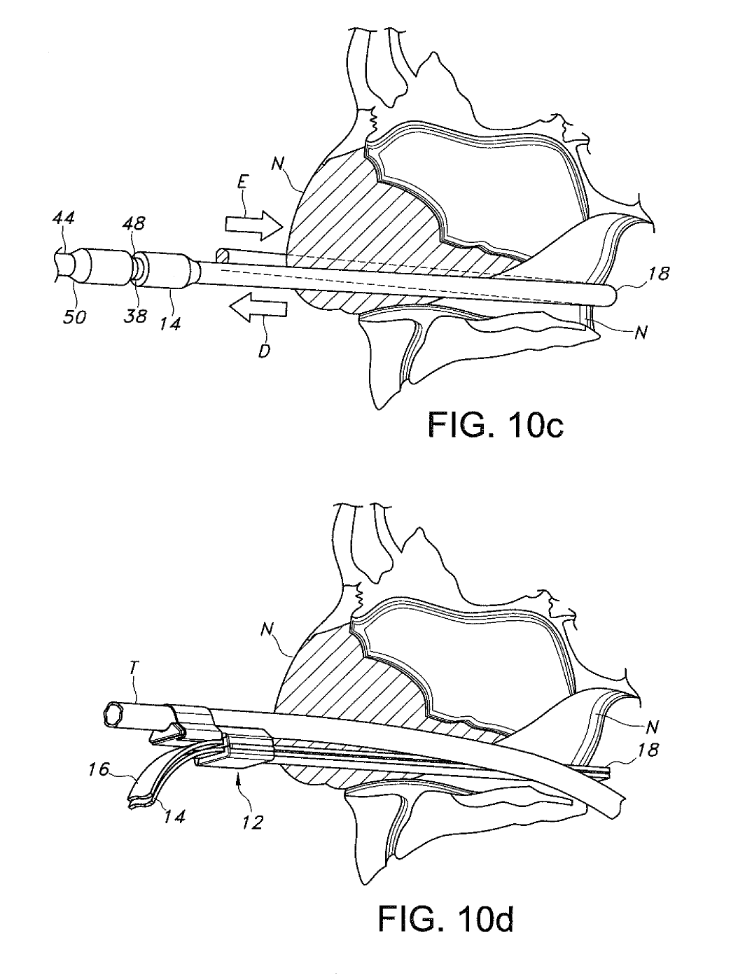

In addition, the overall length of the flexible member 18 should be sufficient to allow a complete loop around the patient's nasal septum N such that end portions 14, 16 of the flexible member 18 extend beyond first and second nares of the patient for securing the nasal tube T as shown in FIG. 10d. The flexible member 18 may further include at least one indicia 42, or mark, or a plurality of marks, indicating an average insertion distance. The distance indicated by the indicia 42 is preferably equal to an average distance from the nares to just beyond the choanal aperture within the nasal cavity. Advantageously, utilization of these marks substantially eliminates unnecessary contact with the posterior wall of the pharynx. For relatively small diameter tubes, a rod or stylet (not shown) may be inserted through a lumen of an end portion of the tube to provide additional rigidity during the insertion process as is known in the art. Depending upon the size and rigidity of the flexible tube, the rigidity of the rod may vary.

With reference to the perspective view of FIG. 4, there is shown a magnetic probe 44 for insertion into a second nare of the patient's nose to attract the magnet 38 and retrieve the end portion 40 of the flexible tube 18. In the present embodiment of the invention, the magnetic probe 44 consists of a rigid or semirigid plastic or rubber cylinder 46 of relatively small diameter having a magnet 48, for example a powerful rare-earth magnet or a ferromagnetic magnet, attached to a first end portion 50. Preferably, the probe 44 is sized for receiving the magnet 48 in a force fit manner or integrally molded around the magnet such that the magnet is secured in an integrated manner within the probe. Of course, other methods of attaching the magnet 48 to the end portion 50 of the magnetic probe 44, such as adhesives, tapes, etc. may be utilized in accordance with the present invention. In order to attract the magnet 38 attached to the end portion 40 of the flexible tube 18, the polarity of the probe magnet 48 is selected to be opposite that of the magnet 38. The length of the magnetic probe 44 must be sufficient to reach from the nare beyond the choanal aperture while still being grasped by the operator's hand or a suitable instrument, and indicia 52 may be provided thereon as well.

As noted above, the first end 16 of the flexible member 18 is securely molded within the receiver 12, or otherwise securely fastened to the receiver, prior to insertion of the second end 14 of the flexible member 18 within the patient. In this manner, the first end 16 is secured by the receiver 12 and the second end 14 is secured by the receiver after insertion within the patient. The second end 14 of the flexible member 18 may be secured between main body portions 22, 24 (within a recess or otherwise) as described above, may be secured along the receiver by an adhesive tape 54 which will be described in more detail below, or may simply be tied to the flexible member 18. Alternatively, both ends 14, 16 of the flexible member 18 may be secured between main body portions 22, 24 (within a recess or otherwise) as described above, or may be secured along the receiver 12 by an adhesive tape 54 which will be described in more detail below.

In the embodiment shown in FIG. 1, the main body 20 of receiver 12 includes a cantilever arm 56 extending there from. The cantilever arm 56 is likewise a molded plastic and has its own arcuate recess formed in a first exterior surface for receiving the tube T. The arcuate recess substantially extends the arcuate recess 26 formed in the main body 20. The arcuate recess formed in the main body 20 and the arcuate recess formed in the cantilever arm 56 will hereinafter be referred to together as arcuate recess 26.

In another embodiment shown in FIG. 5, a portion 57 of main body portion 22 may be extended such that a corresponding portion of the arcuate recess 26 formed thereby near an end 58 of the receiver 12 is formed such that an opening 60 along an axial direction thereof is smaller than an outer diameter of the nasal tube T. In this manner, the tube T may be secured against arcuate recess 26 by snapping the tube T into place. It is contemplated that the portion 57 of main body portion 22 and corresponding portion of the arcuate recess 26 formed thereby is relatively short and intended to initially secure the tube T in the recess until the adhesive tape 54 is applied as described below. The portion 57 of main body portion 22, however, may extend up to the entire length of main body portion 22, or may be positioned near a tip 62 of cantilever arm 56, or at any position between end 58 and tip 62, in accordance with the invention. Advantageously, this significantly simplifies placement of the tube T within the receiver 12 during installation.

As shown in FIG. 2, the receiver 12 further includes an adhesive tape 54 having a first portion 64 secured to the main body 20 and a second portion 66 having a backing 68 thereon covering an adhesive 70. The backing 68 is shown partially cutaway in the figure to reveal the adhesive 70. In this embodiment, the adhesive tape 54 is secured to the cantilever arm 56, and more specifically, the first portion 64 is secured within a slot 72 formed within the cantilever arm.

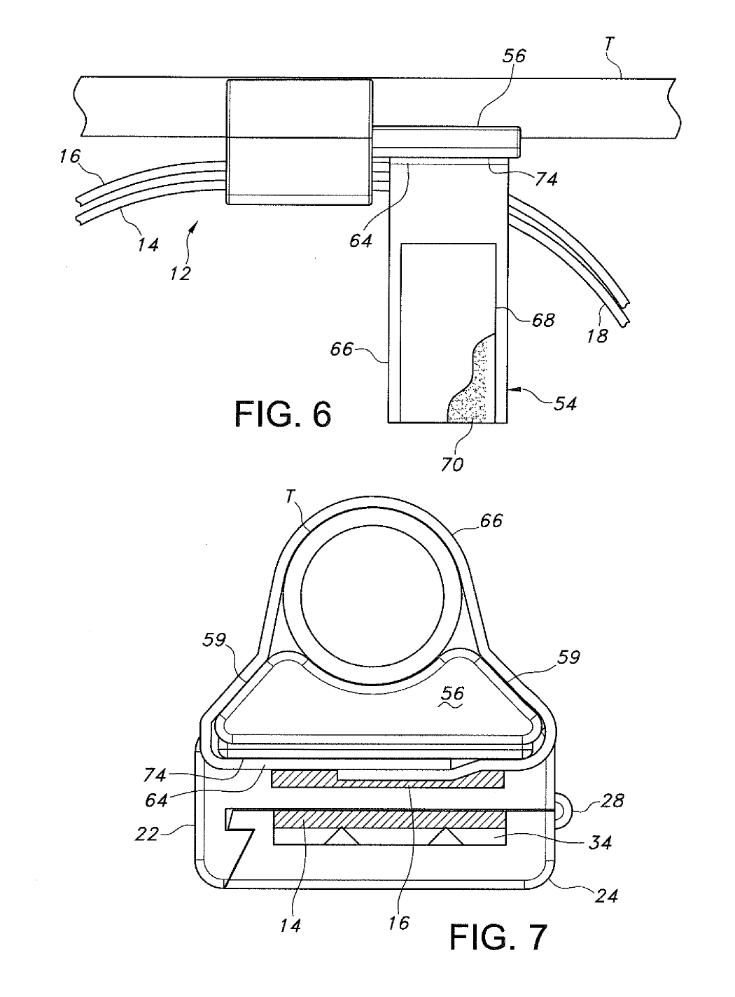

In the embodiment shown in FIG. 6, the adhesive tape 54 is secured to a second exterior surface 74 of the cantilever arm 56. In other words, the adhesive tape 54 is adhered to an underside of the arm 56. In accordance with the invention, the adhesive tape 54 can be secured to any surface or within the main body 20 so long as the second portion 66 of the adhesive tape 54 is able to adhere to the tube T and the main body 20 in order to secure the tube T in position. Alternatively, the second portion 66 of the adhesive tape 54 may adhere to the tube T and the main body 20 in order to secure the tube T in position and at least one, or both, of the end portions 14, 16 of the flexible member 18.

As noted above, FIG. 2 shows the receiver 12 with the first portion 64 of the adhesive tape 54 secured within the slot 72 of the cantilever arm 56, and the second portion 66 of the adhesive tape available to adhere to the tube T and the main body 20 in order to secure the tube T in position. More specifically, the second portion 66 is adhered to the tube T and adjacent surfaces of the cantilever arm 56 on both sides of the tube T in this embodiment. The second portion 66 may even be wrapped around the cantilever arm 56 such that the second portion adheres to the second exterior surface 74 of the cantilever arm.

Alternatively, as shown in FIG. 7, the second portion 66 of adhesive tape 54 may wrap around and adhere to the tube T and adjacent surfaces 59 of the cantilever arm 56 on both sides of the tube T, and the first portion 64 of adhesive tape 54 when the first portion 64 of the adhesive tape is secured to the second exterior surface 74 of the cantilever arm 56 as shown in the FIG. 6 embodiment. More specifically, the first and second portions 64, 66 of adhesive tape 54 may overlap either partially, as shown in FIG. 7, or completely. Although not shown in the figure, the end portions 14, 16 of flexible member 18 may be secured by the second portion 66 of the adhesive tape 54 as described above beneath the cantilever arm 54. Alternatively, the end portions 14, 16 may be secured utilizing the first and second portions 64, 66 of adhesive tape in any position along the main body 20, for example, adjacent the tube T, between the tube T and the arcuate recess 26, adjacent either side of the cantilever arm 56, or adjacent the first portion 64 of the adhesive tape 54, among others.

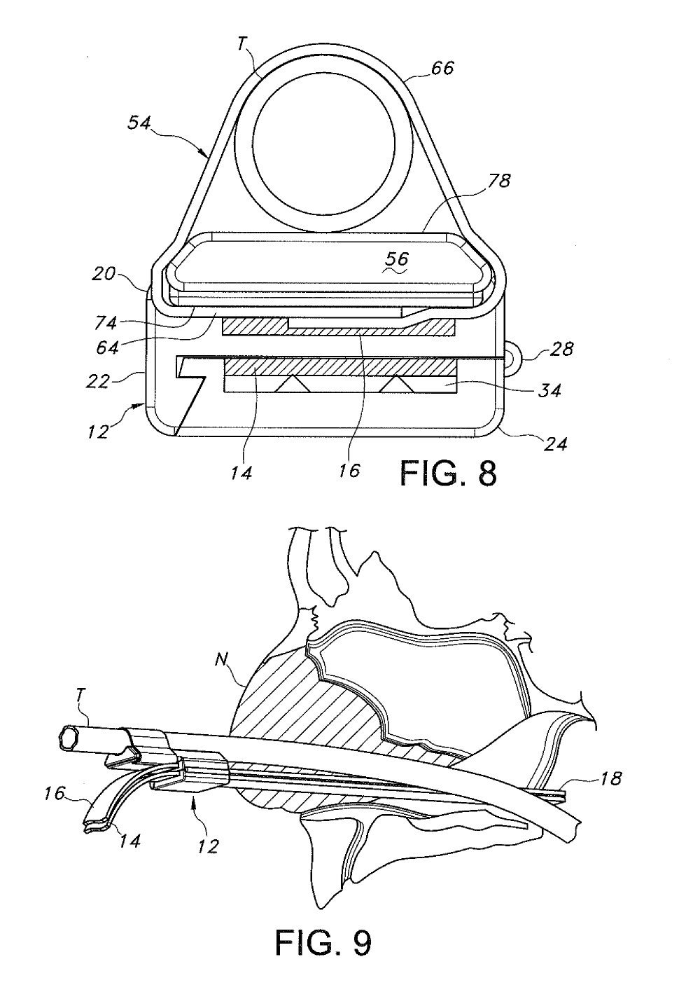

As shown in FIG. 8, the tube T may be positioned along an exterior surface 78 of the main body 20, as opposed to being received within an arcuate recess as shown in FIG. 7. Except for removal of the arcuate recess, the remainder of features of these embodiments is the same as described in FIG. 7. For instance, the receiver 12 includes an adhesive tape 54 having a first portion 64 secured to the main body 20 and a second portion 66 having a backing 68 thereon covering an adhesive 70. The adhesive tape 54 is secured to a second exterior surface 74 of the cantilever arm 56. In other words, the adhesive tape 54 is adhered to the underside of the arm 56.

A method of placing and securing a tube in a patient, as illustrated in FIG. 10, includes the steps of positioning a tube T in a patient such that a portion of the tube extends from the patient, inserting an end portion 14 of a flexible member 18 having a magnet 38 secured thereto into a first nare of the patient's nose, inserting a magnetic probe 44 into a second nare of the patient's nose for attracting the magnet 38, removing the probe 44 from the second nare of the nose thereby retrieving the end portion 14 of the flexible member 18 through the second nare, positioning the portion of the tube T extending from the patient within a recess 26 formed in a receiver 12, and securing the portion of the tube extending from the patient against the receiver using an adhesive tape 54. More specifically, a first portion 64 of the adhesive tape 54 is secured to a first surface of the receiver and a second portion 66 for adhering to the portion of the tube T extending from the patient and a second surface of the receiver.

Prior to insertion into the patient, the tube T, the end portion 14 of the flexible member 18, and the magnetic probe 44 are preferably lubricated with a biologically compatible lubricant. For the comfort of an awake patient, a nasal anesthetic may also be applied. As shown by action arrow B in FIG. 11a, the end portion 14 of the flexible member 18 attached to the magnet 38 is inserted into a first nare of the nose. The flexible member 18 is threaded along a floor of the nose until the end portion 14 of the flexible member 18 and magnet 38 have passed just beyond the posterior border of the nasal septum N through the choanal aperture as shown in FIG. 11b. Preferably, the approximate depth of insertion of the flexible member 18 is indicated by the one or more indicia 42 on the flexible member. Less preferably, the practitioner may rely on resistance encountered when the end portion 14 of the flexible member 18 and magnet 38 reach the posterior wall of the pharynx as an indicator.

At this point, the magnetic probe 44 is similarly inserted into a second nare of the nose as shown by action arrow C in FIG. 11b. The magnetic probe 44 attracts and connects with the magnet 38 attached to the end portion 14 of the flexible member 18 thus allowing the end portion of the flexible member to be retrieved through the second nare of the nose. The magnetic probe 44 is preferably inserted in a substantially parallel course to that of the flexible member 18 along the nasal floor. When the magnetic probe 44 has been inserted to an appropriate depth which may be indicated by indicia 52 on the probe, the magnet 38 attached to the end portion 14 of the flexible member 18 is attracted to the magnetic probe 44, or magnet 48 attached at an end 50 of the probe.

As shown by action arrow D in FIG. 11c, the magnetic probe 44 is then withdrawn from the second nare of the patient's nose, pulling the magnetically coupled flexible member 18 into the first nare and out through the second nare (shown by action arrows E and D in FIG. 11c). Hence, the flexible member 18 is now looped around the nasal septum N posteriorly as shown in FIGS. 11c and 11d.

The magnet 38 of the flexible member 18 and the magnetic probe 44 are then separated and the first tubular portion 14 of the flexible member 18 and magnet 38 are trimmed to a suitable length leaving the second flat portion of the flexible member. The receiver 12 is then secured to the end portions 14, 16 of the flexible member 18 extending from the nares, as well as the nasal tube T, such that a sufficient distance is provided between the nose and the receiver 12 to avoid contact with the patient's nose.

In the present method, the step of securing the tube T includes the step of positioning the tube into an arcuate recess 26 formed in the receiver 12. In an alternate method, the arcuate recess 26, near an end 58 of the receiver, or along its entire length, may be formed such that the opening 60 along an axial direction thereof is smaller than an outer diameter of the nasal tube T (see FIG. 5). In this manner, the tube T may be secured in the arcuate recess 26 by snapping the tube T into place. Of course, in accordance with the broad teachings of the invention, the nasal tube T may be inserted in either nare before or after the above steps are performed. In another alternate method, the tube may simply rest on an exterior surface 78 of the receiver 12 as shown in FIG. 8.

Once positioned adjacent the receiver 12, whether on surface 78 or into arcuate recess 26, the tube T is secured against the receiver using adhesive tape 54. As noted above, a first portion 64 of the adhesive tape 54 may be secured to a first surface 74 of the cantilever arm 54 of receiver 12 and a second portion 66 adheres to the tube T and a second surface of the receiver 59. More specifically, the second portion 66 may adhered to the tube T and adjacent surfaces of the cantilever arm 56 on both sides of the tube T as shown in FIG. 2. The second portion 66 may even be wrapped around the cantilever arm 56 such that the second portion adheres to the second exterior surface 74 of the cantilever arm. Alternatively, as shown in FIG. 7, the second portion 66 of adhesive tape 54 may wrap around and adhere to the tube T and adjacent surfaces 59 of the cantilever arm 56 on both sides of the tube T, and the first portion 64 of adhesive tape 54 when the first portion 64 of the adhesive tape is secured to the second exterior surface 74 (as opposed to being within slot 72) of the cantilever arm 56. More specifically, the first and second portions 64, 66 of adhesive tape 54 may overlap either partially, as shown in FIG. 7, or completely.

In accordance with another aspect of the method, the step of securing includes securing at least one of the end portions 14, 16 of flexible member 18 with receiver 12. As shown in FIG. 2, the end portions 14, 16 of flexible member 18 may be secured between main body portions 22 and 24. More specifically, end portions 14, 16 of flexible member 18 may be secured within recess 34 formed in at least one of the main body portions 22 and 24. The recess 34, as best shown in FIG. 1, may be completely formed in main body portion 22, half formed in main body portion 22 and half in main body portion 24, or more or less than half in each main body portion. Similarly, the shape and/or depth of recess 34 can be any shape or depth so long as one or both of end portions 14, 16 of flexible member 18 can be held securely therein. Even more, the recess 34 is not even required for securing the end portions 14, 16 of flexible member 18 between main body portions 22 and 24. The flexible member 18 may be a generally flat, shoe-string shaped material, a tube-shaped material, or otherwise so long as the material is sufficiently compressible to be held between main body portions 22 and 24.

Alternatively, at least one of the end portions 14, 16 of flexible member 18 may be secured by the second portion 66 of the adhesive tape 54 as described above beneath the cantilever arm 54 adjacent slot 72. Or, the end portions 14, 16 may be secured utilizing the first and second portions 64, 66 of adhesive tape in any position along the main body 20, for example, adjacent the tube T, between the tube T and the arcuate recess 26, adjacent either side of the cantilever arm 56, or adjacent the first portion 64 of the adhesive tape 54, among others.

The foregoing description of the preferred embodiment of the invention has been presented for purposes of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled.

1-49. (canceled)

50. A receiver for securing a tube in a patient and a flexible member positioned around a nasal septum of the patient such that a first end of the flexible member extends from a first nare of the patient and a second end of the flexible member extends from a second nare of the patient, comprising:
a main body securing at least one of the ends of the flexible member and having a first arcuate recess formed in an exterior surface of said main body for receiving the tube; and
a cantilever arm extending from said main body and having a second arcuate recess formed in a first exterior surface of said cantilever arm for receiving the tube, said second arcuate recess substantially extending said first arcuate recess.

51. The receiver of claim 50, wherein said second arcuate recess has an opening, along an axial direction thereof, at least portions of the opening are smaller than an outer diameter of the tube for securing the tube in said recess.

52. The receiver of claim 51, further comprising an adhesive tape having a first portion secured to said main body and a second portion having a backing thereon covering an adhesive, wherein said backing may be removed to expose the adhesive for adhering to at least said main body and the tube.

53. The receiver of claim 52, wherein said first portion of said adhesive tape is secured within said cantilever arm.

54. The receiver of claim 52, where said first portion of said adhesive tape is adhered to a second exterior surface of said cantilever arm.

55. The receiver of claim 51, wherein said first arcuate recess has an opening, along an axial direction thereof, at least portions of the opening being smaller than an outer diameter of the tube for securing the tube in said recess.

56. The receiver of claim 55, further comprising an adhesive tape having a first portion secured to said main body and a second portion having a backing thereon covering an adhesive, wherein said backing may be removed to expose the adhesive for adhering to at least said main body and the tube.

57. The receiver of claim 56, wherein said first portion of said adhesive tape is secured within said cantilever arm.

58. The receiver of claim 56, where said first portion of said adhesive tape is adhered to a second exterior surface of said cantilever arm.

59. The receiver of claim 50, wherein the first end of the flexible member extending from the first nare of the patient is secured within said main body prior to insertion of the second end of the flexible member into the second nare of the patient.

60. The receiver of claim 59, wherein said flexible member is molded within said main body.

61. The receiver of claim 60, wherein said second arcuate recess has an opening, along an axial direction thereof, at least portions of the opening are smaller than an outer diameter of the tube for securing the tube in said recess.

62. A system for placing and securing a nasal tube in a patient, comprising:
a flexible member having first and second ends, said first end for insertion into a first nare of the patient;
a magnet secured at said first end;
a magnetic probe for insertion into a second nare of the patient to retrieve said magnet and said first end of said flexible member; and
a receiver, said receiver having a main body securing at least one of the ends of the flexible member and having a first arcuate recess formed in an exterior surface of said main body for receiving the tube, and a cantilever arm extending from said main body and having a second arcuate recess formed in a first exterior surface of said cantilever arm for receiving the tube, said second arcuate recess substantially extending said first arcuate recess.

63. The system for placing and securing a nasal tube in a patient of claim 62, wherein at least one of said first and second arcuate recesses has an opening, along an axial direction thereof, at least portions of the opening being smaller than an outer diameter of the tube for securing the tube in said recess.

64. The system for placing and securing a nasal tube in a patient of claim 62, wherein the first end of the flexible member extending from the first nare of the patient is secured within said main body prior to insertion of the second end of the flexible member into the second nare of the patient.

65. The system for placing and securing a nasal tube in a patient of claim 64, wherein said flexible member is molded within said main body prior to insertion of the second end of the flexible member into the second nare of the patient.

66. The system for placing and securing a nasal tube in a patient of claim 65, wherein at least one of said first and second arcuate recesses has an opening, along an axial direction thereof, at least portions of the opening being smaller than an outer diameter of the tube for securing the tube in said recess.

67. A method of placing and securing a tube in a patient comprising:
positioning the tube in the patient such that a portion of the tube extends from the patient;
inserting an end portion of a flexible member into a first nare of the patient, said end portion having a magnet secured thereto;
inserting a probe into a second nare of the nose of the patient for attracting said magnet;
removing said probe from the second nare of the nose of the patient thereby retrieving said end portion of said flexible member through the second nare of the nose of the patient;
positioning the portion of the tube extending from the patient within a recess formed in a receiver, said recess formed by a main body and a cantilever arm of a receiver; and
securing the portion of the tube extending from the patient within said recess and against said receiver using an adhesive tape, said adhesive tape having a first portion secured to a first surface of said receiver and a second portion for adhering to the portion of the tube extending from the patient and a second surface of said receiver.

68. The method of placing and securing a tube in a patient of claim 67, wherein said recess has an opening along an axial direction thereof, wherein at least portions of the opening are smaller than an outer diameter of the tube for securing the tube in said recess.

69. The method of placing and securing a tube in a patient of claim 67, wherein the first end of the flexible member extending from the first nare of the patient is molded within said main body prior to insertion of the second end of the flexible member into the second nare of the patient.
